(19) United States
(12) Patent Application Publication
(10) Pub. No.: US 2003/0013105 A1
(43) Pub. Date: Jan. 16, 2003

(54) Methods for identifying drugs specific for known molecular targets using model compounds specific for the molecular targets

(76) Inventors: James D. Thompson, Lafayette, CO (US); Thale C. Jarvis, Boulder, CO (US)

Correspondence Address: SWANSON & BRATSCHUN L.L.C., 1745 SHEA CENTER DRIVE, SUITE 330, HIGHLANDS RANCH, CO, 80129, US

(21) Appl. No.: 10/136,190

(22) Filed: May 1, 2002

Related U.S. Application Data
(60) Provisional application No. 60/287,759, filed on May 1, 2001.

(51) Int. Cl. C12Q 1/68; G01N 33/567

(52) U.S. Cl. 435/6; 435/7.21

(57) ABSTRACT

The present invention provides methods for identifying drugs that are most specific for their intended molecular targets utilizing compounds specific for the molecular targets as model drugs in cultured cells. In various embodiments, methods are described for use of the present invention to identify non-target effects of drugs. The present invention also provides methods to identify other molecular targets for disease intervention besides the intended molecular targets of the drugs. Compounds specific for their molecular targets are preferably antisense agents.

### CROSS-REFERENCE TO RELATED APPLICATIONS

This application claims the benefit of U.S. Provisional Application No. 60/287,759, “Methods for identifying Drugs Specific for Known Molecular Targets Using Antisense Reagents as Model Inhibitors,” filed May 1, 2001, which is incorporated herein by reference.

### FIELD OF INVENTION

The field of this invention relates to methods for determining specificity of drugs to their intended molecular targets in cultured cells using antisense reagents as model drugs, applications of these methods to identify non-target, i.e. non-specific, effects of drugs, as well as applications of these methods to identify other molecular targets that can serve as alternatives to said molecular targets to elicit the desired biological or disease-mitigating effects.

### BACKGROUND OF THE INVENTION

The identification of drugs most specific for their intended molecular targets is a problem of great commercial and human importance. Non-target effects, also referred to as side effects, occur when drugs or their metabolites interact with molecular targets other than their intended targets. For example, nonsteroidal anti-inflammatory drugs act via inhibition of the cyclooxygenase enzyme COX-2 that is induced during inflammatory responses, particularly in macrophages and synovial cells. These drugs, however, can also interact with a closely-related molecular target COX-1 expressed in a wide variety of cell types leading to, among other side effects, gastrointestinal toxicity (see, e.g., Wolfe, et al., Gastrointestinal toxicity of nonsteroidal antiinflammatory drugs. N. Engl. J. Med. 1999;340:1888-1899). As such, second and third generation drugs are now entering the market that have been tailored to be more specific for COX-2 relative to COX-1, with concomitant reduction in side effects.

The importance and necessity of tailoring drugs to specific molecular targets will increase in the future due, in part, to advances in genomics research. Drugs on today's market target approximately 500 molecular targets; however, it is estimated that up to 10,000 molecular targets represent viable drug targets (see, e.g., Drews, Drug Industry: A historical perspective, Science vol 287, March 2000). Discovery of these new drug targets is changing the strategy of drug discovery. Classical drug discovery involves screening compounds in model systems of disease (e.g., cancer drug candidates are screened for inhibition of tumor cell growth in cell culture or reduction of tumor growth in animal models) to identify those compounds that produce a desired biological effect. This screening process often leads to drugs that affect other molecular targets besides those critical for the desired biological effect, leading to undesirable side effects that are not apparent in the screening method used to identify such drugs. In many cases, the molecular targets responsible for the mechanism of action can not be determined by such screening methods. Consequently, the success rate of drugs discovered by this process is low, where less than 10% of drugs entering clinical trials makes it to market, and even drugs that gain regulatory approval may not elicit the optimal combination of potency and specificity (see, e.g., Andersen Consulting “Path to 2008: Key Success Factors for the Pharmaceutical Industry”).

Genomics is changing the strategy in which drugs are discovered. Through genomics, molecular targets critical in a disease process are identified first. Drugs against such validated molecular targets are then selected using screening methods that include the specific molecular target, for example a cloned gene sequence or an isolated enzyme or protein. Typically, such screening strategies produce tens to hundreds of drug candidates capable of interacting with the defined molecular target; however, they tell little about the potential cross-reactivity of these drug candidates to related (known or unknown) molecular targets. This problem can be significant when the molecular target is a member of a large gene family—such as G-protein coupled receptors, protein kinases, proteases and the like—that contain hundreds to thousands of family members. Indeed, to date there has been no improvement in the success rate of drug development using such specific screens involving validated molecular targets. This is due to a lack of reliable methods to distinguish target-specific effects from undesirable non-intended effects. Because of this, drug discovery becomes a trial and error process where compounds with the highest affinity for their molecular targets are advanced into expensive and time-consuming preclinical and clinical studies, only to uncover adverse effects, necessitating repeating the process with other drug candidates. Consequently, there is a need for methods to identify drugs that interact most specifically with their intended molecular targets, and to identify and eliminate those drugs that interact adversely with other non-intended molecular targets.

### SUMMARY OF THE INVENTION

The present invention provides methods involving inhibiting or stimulating a molecular target with antisense reagents in a cell system expressing the molecular target, and evaluating the effect of such modulation using a variety of measurements to generate a model antisense response. Data from the model antisense response are used as the benchmark to evaluate the specificity of drugs intended to interact with the same molecular target. Such drugs may be administered to the same cell system to generate a drug response and the resulting changes in the function of the molecular target(s) compared with those of the model antisense drugs. Changes in the function of the molecular target can be measured by direct or indirect methods and can include changes in cell phenotype, the transcriptome, the metabolome and/or the proteome. In one embodiment, the methods of the present invention can be used to identify the drug having the highest specificity for the intended molecular target by determining the specificity of at least two drugs and comparing the specificities. In another embodiment, the invention provides a method to identify at least one non-intended effect of a drug, otherwise known as a drug side effect, by comparing the model antisense response with the drug response, and detecting a difference. In another embodiment, the invention provides a method to identify at least one non-intended effect of a drug in a system which does not express the molecular target by comparing the model antisense response with the drug response, and detecting differences. In another embodiment, the invention provides a method to identify a non-intended effect of a drug by generating a combined antisense and drug response, comparing the combined response to a drug response, and detecting a difference. In yet another embodiment, the present invention provides a method to identify molecular targets whose function may be modified to produce a desired biological effect by comparing a model antisense response with an antisense response generated by at least one other antisense reagent that affects a secondary target, and comparing the responses. In another embodiment, the invention provides a method for refining the determination of drug specificity for a protein molecular target by measuring an antisense response for a system having a homolog of the protein using at lease one model antisense reagent, measuring a drug response for the system having the homolog, and comparing the responses. In a further embodiment, the invention provides a method to determine differences in drug responses in different systems by measuring an antisense response, measuring a drug response in a cell system from a different species, and detecting a difference. In another embodiment, the invention provides a method to determine the effect of combining more than one drug and comparing said response to the combined drugs with the model antisense response to identify drug combinations that provide the desired biological effect.

### DETAILED DESCRIPTION OF THE PREFERRED EMBODIMENTS

This invention provides methods for utilizing a target-specific compound as a drug model for an intended molecular target in order to determine the specificity of a drug intended to modify the function of that intended molecular target. In a preferred embodiment, the target-specific compound is an antisense reagent.

The methods of the present invention comprise modulating the function of a molecular target with target-specific agents in a cell system expressing the molecular target, and evaluating the effect of such modulation using a variety of measurements to generate a model response. By “modulating the function” or “modulating the activity” it is meant altering when compared to not adding an agent. Modulation may occur on any level that affects function. A polynucleotide or polypeptide function may be direct or indirect, and measured directly or indirectly. Modulation may be an increase (stimulation) or a decrease (inhibition) in the function of the target. Data from the model response are used as the benchmark to evaluate the specificity of drugs intended to interact with the same molecular target. Such drugs may be administered to the same cell system to generate a drug response and the resulting changes in the function of the molecular target(s) compared with those of the model target-specific agents. Evaluating the effects can be accomplished by direct or indirect methods and can include detecting changes in cell phenotype, the transcriptome, the metabolome and/or the proteome. By comparing model responses with drug responses in various systems, drug specificity, non-target or side effects, and cell system-specific effects, inter alia, can be determined.

It is to be noted that the term “a” or “an” entity refers to one or more of that entity; for example, a target-specific compound refers to one or more target-specific compounds. As such, the terms “a” (or “an”), “one or more” and “at least one” can be used interchangeably herein. Although the use of antisense technology to practice the methods of the invention is described herein, it is to be understood that any target-specific compounds can be used. Target-specific compounds also include, but are not limited to, proteins, including but not limited to antibodies, zinc finger binding proteins, and proteins which mediate RNA editing; nucleic acids, including but not limited to aptamers, ribozymes, chimeraplast molecules, and small interfering RNA (siRNA); cofactors, including but not limited to ATP and NAD; lectins; enzymes; carbohydrates; receptors and receptor ligands; heparin, and viruses. Antibodies can include anti-sera containing antibodies, or antibodies that have been purified to varying degrees. Antibodies include functional equivalents such as antibody fragments and genetically-engineered antibodies, including single chain antibodies, that are capable of selectively binding to at least one of the epitopes of the target. Antibodies that may be used in the present invention also include chimeric antibodies that can bind to more than one epitope. Aptamer, as used herein, includes nucleic acid molecules that bind to specific non-nucleic acid molecular targets, such as a protein or metabolite. Chimeraplast, as used herein, refers to a synthetic nucleic acid molecule capable of directing repair of base pair mutations, deletions or insertions. siRNA is a homologous double stranded RNA that specifically target a gene's product, resulting in null or hypomorphic phenotypes.

As used herein, “antisense technology” in its most general form refers to the use of a collection of nucleotide sequences which are not templates for synthesis but yet interact with complementary sequences in other molecules thereby causing a function of those molecules to be affected. As used herein, “complementary” refers to nucleic acid base sequences that can form a double-stranded structure by matching base pairs. Matching base pairs are formed by way of a regular pattern of monomer-to-nucleoside interactions such as Watson-Crick type of base pairing, Hoogsteen or reverse Hoogsteen types of base pairing, or the like. Antisense technology includes affecting the functions of DNA, including replication and transcription through the use of antisense reagents. Also included is affecting the functions of RNA, including all vital functions, such as, for example, translocation of the RNA to the site of protein translation, translation of protein from the RNA, splicing of the pre-mRNA to yield one or more mRNA species, guide RNAs acting as templates for other RNA modifications or editing, catalytic activity which may be engaged in or facilitated by the RNA, structural integrity of the RNA (e.g., facilitating cleavage of the RNA), or stability or half-life of the RNA. The overall effect of such interference with target nucleic acid function is modulation of the expression of a gene (and its corresponding gene product or protein). In the context of the present invention, inhibition is the preferred form of modulation of gene expression and RNA is a preferred antisense target.

As used herein, a “molecular target” refers to any cell component whose function is modified by interaction with a drug. Molecular targets include proteins, nucleic acids, lipids or other intracellular or extracellular components. Preferred molecular targets are proteins and nucleic acids. In particularly preferred embodiments, molecular targets may include but are not limited to cyclooxygenase, steroidal receptors (e.g., androgen receptor, estrogen receptor, glucocorticoid receptor), non-steroidal receptors (e.g., insulin receptor, nerve growth factor receptor, TNF-alpha receptor, IL-2 receptor, interleukin receptors, beta-adrenergic receptors, angiotensin receptor), neuronal receptors (e.g., serotonin receptor, dopamine receptor, GABA receptor), H+/K+ ATPase proton pump, calcineurin, metabolic enzymes (e.g. IMPDH-II, HMG-CoA reductase, COX-2, ACE,), ion channels (e.g. calcium channel), protein kinases (e.g., AKT-1), protein phosphatases (e.g., PP2), proteases (e.g. angiotensinogen). In a preferred embodiment, molecular targets are those classical drug targets described in Drews & Ryser, 1997, Classic drug targets, Nature Biotechnology 15:special pullout. This reference, and all other patent and publications referred to herein, are incorporated by reference herein in their entirety. A “drug”, as used herein, in its most general form, is a substance used in the diagnosis, treatment, or prevention of a disease or as a component of a medication, or is any compound that affects the function of a biological system. Pharmaceutical compositions comprising more than one drug are within the scope of this invention. The molecular target of a drug may be known or unknown, intended or unintended. Often, the intended molecular target for a drug is only one actual molecular target for such drug. Additionally, drugs may have primary and secondary molecular targets. For example, a given drug may inhibit the function of a first protein. The inhibition of the first protein, in turn, may suppress the expression of a second protein. In this way, the first protein is a primary target of the drug, and the second protein is the secondary target of the drug. Molecular targets, as used herein, include primary secondary, tertiary, etc., molecular targets.

Antisense reagents may be employed to affect the function of molecular targets. In the case of a nucleic acid molecular target, the molecular target is generally a primary target for the antisense reagent, and will be referred to as a primary antisense target. In the case of a protein molecular target, the molecular target is a secondary (or tertiary, etc.) antisense target for the antisense reagent. Those skilled in the art will recognize that an antisense reagent, by definition, can not affect a protein target directly. Rather, the antisense reagent affects the function of the protein by stimulating or inhibiting gene expression, protein translation, or performing some other antisense effect (see, e.g., Crooke 1999, Molecular mechanisms of action of antisense drugs. Biochim Biophys Acta December 10;1489(1):31-44; Matteucci 1997, Oligonucleotide analogues: an overview. Ciba Found Symp 1997;209:5-14). Thus, while a protein may be a primary molecular target for the drug, it will be a secondary antisense target for the antisense reagent.

Antisense reagents used as part of the present invention as a drug model typically affect the expression of their intended molecular targets to a large degree, and are termed model antisense reagents. In one embodiment, the model antisense reagents affect the function of their intended molecular target by greater than or equal to 50%. In another embodiment, the model antisense reagents affect the function of the intended molecular target by greater than or equal to 70%. In another embodiment, the model antisense reagents affect the function of the intended molecular target by greater than or equal to 85%. In another embodiment, the model antisense reagents affect the function of the intended molecular target by greater than or equal to 90%. In another embodiment, the model antisense reagents affect the function of the intended molecular target by greater than or equal to 95%. In another embodiment, the model antisense reagents affect the expression of the intended molecular target by greater than or equal to 99%.

Preferably, an antisense reagent of the present invention is a synthetic nucleic acid of at least 6 nucleotides in length. In preferred embodiments, an antisense oligonucleotide is at least about 10 nucleotides, at least about 15 nucleotides, at least about 25 nucleotides, or at least about 100 nucleotides in length. The antisense reagent can be DNA or RNA or chimeric mixtures or derivatives or modified versions thereof and can contain single stranded or double stranded regions. The antisense oligonucleotide can be modified at the base or sugar moieties or the phosphate backbone.

In a preferred embodiment of the invention, an antisense reagent is a chimeric oligonucleotide containing RNA and DNA or derivatives thereof that provide minimal effects on non-intended targets in the cell system while providing the desired level of modulation of the intended molecular target. Effects on non-intended targets, herein referred to as antisense sideeffects, are determined by contacting the cell system with substantially similar doses and formulations of negative control antisense reagent comprising, but not limited to, either a single antisense reagent or heterogeneous mixtures of different antisense reagents with substantially similar chemical compositions or derivatives as the model antisense reagents against the intended target, but targeting either different targets or no targets, and measuring the cellular response of the cell system to generate a negative control antisense response. In another preferred embodiment, chemical modifications include those that produce the least number of antisense side effects.

Oligonucleotide derivatives may comprise any of a number generally known in the art and may include at least one modified base moiety selected from the group including, but not limited to 5-bromouracil, hypoxanthine, xanthine, inosine, 1-methyl guanine, 2,2-dimethylguanine, 5-methylcytosine, 7-methylguanine, 4-acetylcytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, N⁶-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N⁶-methyladenine, 7-methylguanine, 5-methylaminomethyluracil, 5-methoxyaminomethyl-2-thiouracil, β-D-mannosylqueosine, 5-methoxycarbonylmethyluracil, 5-methoxyuracil, 2-methylthio-N⁶-isopentenyladenine, uracil-5-oxyacetic acid methylester, uracil-5-oxyacetic acid (v), wybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, and 2,6-diaminopurine.

In another embodiment, the antisense reagent contains modified 3′-terminal internucleotide linkages which confer resistance to 3′-5′ exonucleolytic degradation, selected from the group including but not limited to 3′-3′ inverted sugars or nucleotides, biotin, phenyl, naphthyl, and phosphotriester. In another embodiment, the antisense reagent contains modified 5′-terminal internucleotide linkages which confer resistance to 5′-3′ exonucleolytic degradation, selected from the group including but not limited to 5′-5′ inverted sugars or nucleotides, biotin, phenyl, naphthyl, and phosphotriester. In another embodiment, the antisense reagent comprises at least one modified sugar moiety selected from the group including, but not limited to, arabinose, 2′-O-methylribose, 2′-fluoroarabinose, 2′-methoxyribose, 2′-ethoxyribose, and 2′-methoxyethoxyribose. In yet another embodiment, the antisense reagent comprises at least one modified phosphate backbone selected from the group consisting of a phosphorothioate, a phosphorodithioate, a phosphoroamidiothioate, a phosphoramidate, a phosphoridamidate, a P-ethoxyphosphodiester, a methylphosphonate and an alkyl phosphotriester. An antisense reagent can include a non-nucleic acid group such as a peptide, a lipid, a fluorophore or other non-nucleic acid moiety that improves intracellular stability or facilitates transport across cellular membranes or affects intracellular localization or otherwise improve the potency or specificity of the reagent. Antisense agents are preferably optimized for delivery in the target cell type. Antisense reagents can enter cultured cells when administered directly to the cell culture media (see, e.g., Heikkila et al., 1987, Nature 328:445-449); however, various delivery methodologies are commonly used by those skilled in the art to improve efficiency and consistency of delivery of antisense reagents to appropriate intracellular compartments. For example, antisense reagents may be delivered to adherent cells in culture using lipid carriers as described in Jarvis et al., 1996, “Inhibition of vascular smooth muscle cell proliferation by ribozymes that cleave c-myb mRNA.” RNA 2: 419-428, and in Jarvis et al., 2000, “Ribozymes as tools for therapeutic target validation in arthritis” J Immunol. 165:493-8. Antisense reagents (final concentration 6-200 nM) and an appropriate cationic lipid delivery vehicle such as LipofectAMINE (Life Technologies, Inc. final concentration 1-16 μg/ml) may be combined in complete media, incubated at 37° C. for 30 mins in polystyrene tubes to form antisense/lipid complexes. Complexes may then be added to cells in a 1:1 ratio of cell culture media and lipid/antisense complexes. Complexes may be left on cells for the duration of the experiment (typically 1-5 days). Delivery methodologies can include, but are not limited to, electroporation or calcium phosphate co-precipitation (see e.g. Sambrook et al., Molecular Cloning: A Laboratory Manual, Cold Spring Harbor Labs Press, 1989), use of pore-forming proteins (e.g., Streptolysin-O, Sigma Corp.), attachment to antisense reagents of carrier molecules such as transferrin that are normally taken up by cells, and use of lipid carriers such as LipofectAMINE (Life Technologies, Inc.). Antisense reagents may be delivered to cells grown in suspension cultures, such as blood cells, using a modified centrifugation-based transfection protocol (see, e.g. Verma et al., 1998, “Increased efficiency of liposome-mediated transfection by volume reduction and centrifugation.” BioTechniques, 25:46).

In a preferred aspect of the invention, delivery methodologies are chosen for each cell type that have minimal effect on the biology of the cells, in particular but not limited to, toxicity. Toxicity can be measured by a number of methods known to those skilled in the art such as trypan blue exclusion, propidium iodide exclusion, MTS assays for mitochondrial activity (e.g. CellTiter 96 Aqueous Non-Radioactive Cell Proliferation Assay, Promega Inc.), or inhibition of cell proliferation as measured by direct counting of cells or by commercially available kits (e.g., CyQUANT Cell Proliferation Assay Kit, Molecular Probes). In a preferred aspect of the invention, optimal delivery methodologies and conditions are evaluated by comparing efficacy of a positive control antisense reagent to toxicity of a negative control antisense reagent. A positive control antisense reagent can be an antisense reagent known to inhibit a cellular RNA, e.g. an antisense reagent targeting commonly expressed mRNA such as c-Raf (see, e.g.,—Monia et al., 1996, Nature Medicine 2:668-75). Negative control antisense reagents can include, but are not limited to, antisense reagents comprising substantially similar chemical modifications as the positive control antisense reagent and comprised of sequences that are substantially similar to the control antisense reagent but contain 1-7 mismatches in positions that reduce or completely inactivate the activity of the positive control antisense, the sense sequence of the positive control antisense reagent, the reverse sequence of the positive control antisense reagent, a randomized sequence (i.e. a mixture of all possible sequences) or a scrambled sequence of the positive control antisense (see, e.g. Agrawal and Kandimalla, 2000, “Antisense therapeutics: is it as simple as complementary base recognition?” Molecular Medicine Today, 6:72-81). In a preferred aspect of the present invention, optimal delivery conditions are determined by comparing inhibition of the target mRNA obtained with the positive control antisense reagent to toxicity produced with the negative control antisense reagent under substantially similar delivery conditions. Ideal delivery conditions represent delivery methodologies and antisense reagent doses that produce the greatest reduction of the intended target RNA level achieved by the positive control antisense reagent, with minimal toxicity observed with the negative control antisense reagent.

In general, antisense reagents that are complementary to the intended target are designed and optimal reagents are identified empirically by testing a number of antisense reagents designed to bind to the intended molecular target using optimal delivery and dosing conditions to identify reagents most effective in modulating the molecular target (e.g., see Monia et al., 1996, ibid.). In instances where the molecular target is a messenger RNA (mRNA), and the desired biological effect is reduction of the said target mRNA, reduction of the said target mRNA may be measured using any of a number of assays known to those skilled in the art including, but not limited to, Northern Blotting, RNase protection assays, primer extension (see, e.g. Sambrook et al., ibid), or QC-PCR (e.g. TaqMan assays, Applied Biosystems, Inc.). Antisense reagents may be designed to bind to the 5′ untranslated region, protein coding region or 3′ untranslated region of the target mRNA, intronic sequences of the precursor hnRNA of the target mRNA, exon-intron junctions, or the translational start site. It is preferable that antisense reagents lack motifs known to produce non-specific effects, where such motifs comprise CpG DNA dinucleotides, G quartets and other features that produce non-specific effects as described in Agrawal and Kandimalla, 2000, ibid. In another preferred aspect of the invention, antisense sequences are further filtered to remove those that exhibit homology to other sequences besides the intended molecular target. Sequences exhibiting homology to other, non-intended target sequences, may be identified by those skilled in the art using sequence comparison programs including, but not limited to, the BLAST program available through the web site of the National Center for Biotechnology Information. In a preferred aspect of the invention, ideal antisense sequences will be less than 90% homologous, 80%, 70%, 60% or 50% to other ESTs, mRNAs or other nucleic acid sequences contained in public databases such as Unigene, dbEST, Genbank and the like, or proprietary databases such as LifeSeq (Incyte Genomics) or Celera Discovery System (Celera). In another preferred aspect, sequences will contain less than 16 nucleotides of contiguous homology to non-intended targets.

Antisense reagents may be synthesized by standard methods known in the art, e.g. as in Wincott, et al., 1995, Synthesis, deprotection, analysis and purification of RNA and ribozymes, Nucl Acids Res. 23: 2677, see also the following monograph “Oligonucleotide synthesis: A practical approach (Gait M. J., ed.) IRL Press, Oxford (1984). Alternatively, antisense reagents can be obtained from commercial vendors including but not limited to, Integrated DNA Technologies, Inc., Oligos Etc., Inc., Life Technologies, Inc, TriLink, Inc. Midland Corporation and the like.

An antisense reagent may comprise a single oligonucleotide chosen from a number of target-specific antisense oligonucleotides and exhibiting the desired level of modulation of the molecular target. In other embodiments, an antisense reagent may comprise a mixture of at least two antisense reagents, at least four antisense reagents or at least eight antisense reagents which, as an admixture, achieve the desired level of modulation of the molecular target.

In one embodiment, the present invention provides methods involving inhibiting or stimulating a molecular target with antisense reagents in a cell system expressing the molecular target, and evaluating the effect of such modulation using a variety of measurements. As used herein, “cell system” refers to any in vitro culture of cells. Included within this term are continuous cell lines (e.g., with an immortal phenotype), primary cell cultures, finite cell lines (e.g., non-transformed cells), tissue explants, and any other cell population maintained in vitro. The system may comprise a discrete cell lineage, a mixture of cell or tissue types, and cells in various or specific stages of differentiation. As used herein, the term “in vitro” refers to an artificial environment and to processes or reactions that occur within an artificial environment. An in vitro environment comprises, but is not limited to, a test tube or cell culture. The term “in vivo” refers to the natural environment (e.g., an animal or a cell) and to processes or reactions that occur within a natural environment. Preferred cell systems include those derived from human tissues. Particularly preferred cell systems include those derived from brain (neuronal, e.g., American Type Culture Collection [ATCC] #CRL-10442; neural progenitor cells, e.g., Clonetics #CC-2599), heart (normal aorta smooth muscle, e.g., ATCC #CRL-1999; normal coronary artery smooth muscle, e.g., Clonetics #CC-2583; cardiomyocytes), lung (normal lung epithelial, e.g., ATCC #CRL-9442; normal lung fibroblast, e.g., Clonetics #CC-2512), kidney (embryonic epithelial, e.g., ATCC #CRL-1573; normal cortical epithelial cells, e.g., Clonetics #CC-2554), liver (epithelial e.g., Hep G2 cells, ATCC #HB-8065; normal hepatocytes, e.g., Clonetics #CC-2695), bone (osteosarcoma or chondrosarcoma, e.g., ATCC #'s HTB-96 or HTB-94 respectively; normal osteoblasts or chondrocytes, e.g., Clonetics #'s CC-2538 or CC-2550 respectively), skin (normal fibroblast or Keratinocytes, e.g., ATCC #'s CCL-110 or CRL-2404; normal epidermal keratinocytes, e.g., Clonetics #CC-2501), GI tract/colon (gastric, e.g., ATCC #CRL-1739; normal colon smooth muscle, e.g., Clonetics #CC-2573; gastric parietal cells), mammary (epithelial, e.g., ATCC #HTB-22; normal mammary epithelial, e.g., Clonetics #2551), prostate (epithelial, e.g., ATCC #CRL-1740; normal prostate epithelial or fibroblastic, e.g., Clonetics #'s CC-2555 and CC-2508 respectively), endocrine (pancreatic, e.g., ATCC #CRL-1469; adrenal, e.g., ATCC #CCL-105; thyroid, e.g., ATCC #CRL-1803), cervix (epithelial, e.g., ATCC #CCL-2; normal cervical epithelial, e.g., Clonetics #CC-2648), ovarian (epithelial, e.g., ATCC #HTB-161), or mouse adipose tissue (e.g., ATCC #CCL-1).

Evaluating the effect of the modulation of the molecular target is referred to herein as “measuring the cellular response.” Measuring the cellular response includes, but is not limited to, evaluation of changes in cell phenotype, the transcriptome, metabolome and/or the proteome. As used herein, “phenotype” refers to any of the observable physical, behavioral, morphological or biochemical characteristics of a cell or cell system, including the expression of a specific trait, based on genetic and environmental influences. As used herein, “transcriptome” refers to the make up, variety and abundances of RNA transcripts expressed in a cell system. As used herein, “metabolome” refers to the chemical make-up of a cell or cell system, including but not limited to variety and intracellular/extracellular concentrations of all metabolites involved in metabolic processes and organelle structure or composition (see, e.g., Raamsdonk et al., 2001, “A functional genomics strategy that uses metabolome data to reveal the phenotype of silent mutations” Nature Biotechnology 19:45-50). As used herein, “proteome” refers to the make up, variety, abundances, modifications and activities of proteins expressed in or secreted by a cell system. Evaluating the effect of the modulation of the molecular target is referred to herein as “measuring the cellular response.”

In order to generate an antisense response, cells are treated either with no reagents (i.e. untreated) or positive control antisense and negative control antisense and varying doses of antisense reagents, with or without delivery vehicle, on day 0 and harvested at varying times post-treatment. In a preferred embodiment, samples are harvested at 4 hours, 8 hours, 1 day, 2 days, 3 days, 4 days and 5 days post-treatment and cellular responses measured. The pretreatment and post-treatment cell culture protocol may include additional stimuli of importance for maintaining or promoting the relevant biological phenotype of the cells, including but not limited to addition or removal of serum, addition or removal of cytokines, addition or removal of reagents that induce or relieve cell-cycle arrest, addition or removal of conditioned medium from other cultured cells or tissue explants, addition or removal of biological fluids, changes in temperature or other environmental conditions of culture.

Data from the model antisense response are used as the benchmark to evaluate the specificity of drugs intended to interact with the same molecular target. Such drugs may be administered to the same cell system and the resulting changes in the function of the molecular target(s) compared with those of the model antisense drugs.

Generation of a drug response is similar to the generation of an antisense response. In parallel to antisense treatment of cells, the same cell systems are treated with candidate drugs of interest at varying doses. In a preferred embodiment, the dose range spans about 0.1× below to about 100× above the IC₅₀ (inhibitory concentration) or ED₅₀ (effective dose) for the drug in the cell system. IC₅₀ is defined as the dose at which the drug inhibits its intended molecular target or biological phenotype 50% relative to the untreated cells. Alternatively, the relevant dose range may span below and above the ED₅₀, where ED₅₀ is defined as the dose at which the drug elicits a biological response 50% relative to untreated cells. Cells are treated with drug on day 0 and harvested at varying times post-treatment. In a preferred embodiment, samples are harvested 1 hour, 2 hours, 4 hours, 8 hours, 1 day, 2 days, 3 days, 4 days and 5 days post-treatment and cellular responses measured.

Changes in the function of the molecular target can be measured by direct or indirect methods and can include changes in cell phenotype, the transcriptome, the metabolome and/or the proteome. Relevant molecular targets may include both intracellular, cell surface or secreted entities. Changes in phenotype include but are not limited to changes in differentiation state of a cell system, changes in proliferative capacity of the cell system (e.g., induction of cell proliferation, changes in rate of proliferation, growth arrest in a particular stage of the cell cycle such as G1, S, G2 or mitosis), cellular toxicity, induction or suppression of apoptosis, induction or supression of cell motility and gross changes in cell morphology.

Changes in the transcriptome include but are not limited to changes in any portion of the transcriptome. Changes in the transcriptome can by measured by a variety of techniques known in the art such as Northern blotting, RNase protection and primer extension, and other commercially available technologies including, but not limited to, QC-PCR (e.g., TAQMAN® technology with instrumentation and reagents commercially available from Applied Biosciences, Inc., Foster City, Calif.), microarray technology and instrumentation offered by such commercial vendors as Affymetrix, Santa Clara, Calif., Agilent Technologies, Palo Alto, Calif., Incyte Genomics, Palo Alto, Calif. and the like, differential display (e.g., see products and services offered by Digital Gene Technologies, La Jolla, Calif.), and SAGE (e.g., see products and services offered by Genzyme Molecular Oncology, Framingham, Mass., and Invitrogen, Inc., Carlsbad, Calif.).

Changes in the metabolome (see, e.g., Raamsdonk et al, 2001, ibid) include but are not limited to changes in levels of amino acids, nucleotides and nucleosides, sugars, cAMP, and the like. Changes in the metabolome can be measured by a variety of techniques including but not limited to electrospray mass spectrometry (ES-MS), liquid chromatography mass spectrometry (LC-MS), Fourier-transform infrared spectroscopy (FTIR), nuclear magnetic resonance (NMR), and two dimensional thin layer chromatography (2D TLC).

Changes in the proteome include but are not limited to changes in any portion of the proteome. Changes in the proteome can by measured by a variety of techniques known in the art such as Western blotting, FACS analysis of proteins in whole or fixed cells, immunoprecipitation, ELISA measurement of proteins in fixed cells, cell lysates and supernatants, and other commercially available technologies including, but not limited to, antibody arrays, 2-dimensional gel electrophoresis, aptamer arrays, activity measurements performed by functional, biochemical or physical means such as mobilization of intracellular calcium, covalent modification of select proteins, activation of transcription factors as measured by gel shift assays, other measurements of protein binding interactions such as affinity chromatography, radioligand binding, two-hyrbrid systems, and the like.

In one embodiment, this invention provides a method to determine the specificity of at least one drug for a molecular target. Specificity can be measured in a number of ways known in the art. In one embodiment, specificity can be expressed as a percentage of the similarity of the drug cellular response relative to the model antisense response in cell systems expressing the target, where:

Per Cent Specificity=100%×(M)/(C) (Equation 1)

where M is the sum of matches between antisense and drug responses, and C is the sum of total changes observed with antisense and drug responses. The following example represents one of a number of possible methods to evaluate specificity of a drug response relative to the antisense benchmark response to arrive at a specificity value for Equation 1. In this example, the “state” of 20 intracellular constituents, referred to as X₁ through X₂₀, are used to measure the cellular response, where “state” refers in this example to the abundance of specific RNAs designated as X₁ through X₂₀ (X₁ through X₂₀ could also refer to other measurements of the transcriptome, cell phenotype, metabolome, proteome or any combination of these). In this example, the abundance of transcripts X₁ through X₂₀ are measured using microarrays, although Northern blotting, QC-PCR or the like could also be used. Cellular responses are measured in a cell system that is untreated (untreated response), a substantially similar cell system treated with antisense reagent (antisense response) and a substantially similar cell system treated with drug (drug response). Table I shows hypothetical results.

TABLE I
Hypothetical specificity analysis

| A | B | C | D | |
|---|---|---|---|---|
| Cellular | Antisense vs | Drug vs. | Change in | E |
| constituent | Untreated | Untreated | antisense or drug | Changes match |
| X₁ | 0 | 0 | 0 | |
| X₂ | 1 | 1 | 1 | 1 |
| X₃ | 1 | 1 | 1 | 1 |
| X₄ | −1 | 1 | 1 | 0 |
| X₅ | 1 | 0 | 1 | 0 |
| X₆ | 1 | −1 | 1 | 1 |
| X₇ | 0 | 0 | 0 | |
| X₈ | 1 | 0 | 1 | 0 |
| X₉ | 0 | 1 | 1 | 0 |
| X₁₀ | −1 | 1 | 1 | 0 |
| X₁₁ | 0 | −1 | 1 | 0 |
| X₁₂ | 0 | 1 | 1 | 0 |
| X₁₃ | 0 | 0 | 0 | |
| X₁₄ | −1 | −1 | 1 | 1 |
| X₁₅ | 0 | 0 | 0 | |
| X₁₆ | −1 | −1 | 1 | 1 |
| X₁₇ | 0 | 0 | 0 | |
| X₁₈ | 1 | 1 | 1 | 1 |
| X₁₉ | 0 | 0 | 0 | |
| X₂₀ | −1 | 1 | 1 | 1 |
| Total | | | 14 | 7 |

A change in the state of X₁ through X₂₀ RNAs in either the antisense or drug treated cell systems is determined by comparing the values obtained from the microarray analysis of these samples relative to the untreated sample. Three values are possible: if no significant change is observed in the state of X₁ through X₂₀ in antisense and drug responses relative to the untreated sample, then a value of “0” is assigned to that constituent; if a statistically significant increase in the abundance of X₁ through X₂₀ RNAs is detected in the antisense and drug responses relative to the untreated sample, then a value of “+1” is assigned; similarly, if a statistically significant decrease in the abundance of X₁ through X₂₀ in antisense and drug responses relative to the untreated sample is observed, then a value of “−1” is assigned (see, Table I, Columns B & C). Column D in Table I indicates if a change of state of X₁ through X₂₀ RNAs occurred in either the antisense or drug responses relative to the untreated; if a change of state occurred in either of these samples, then a value of “1” would be assigned, if no change of state occurred, then a value of “0” would be assigned. A match between the antisense response and the drug response would be recorded if the values were not zero in column D and the values in columns B&C matched (see, Column E of Table I). The sum of matches between antisense and drug responses, or M, in equation 1 can be found by summing column E in Table I. The sum of total changes observed with antisense and drug responses, or C, can be found by summing column D in Table I. Applying these values to equation 1 yields a Per Cent Specificity of 100%×(7/14) or 50%. In one embodiment, this data analysis could include more precise gradations of response for both increases and decreases in levels of cellular constituents in order to score the matching of the values of each constituent for antisense and drug-treated samples. In another preferred embodiment, the analysis might include statistical algorithms known to those skilled in the art including, but not limited to, hierarchical clustering, self-organizing maps, divisive clustering and k-means clustering to assist in pattern recognition and matching of cellular response profiles (see, e.g., Sherlock 2000, Analysis of large-scale gene expression data.Curr Opin Immunol April;12(2):201-5; and Reibnegger Wachter 1996, Self-organizing neural networks—an alternative way of cluster analysis in clinical chemistry.Clin Chim Acta April 15;248(1):91-8).

In one embodiment, this invention provides a method to identify non-target or side effects of a drug, where:

Non-target drug effects=C−M (Equation 2)

where C and M are defined as in Equation 1. In the present example, the C can be found by summing column D in Table I and M can be found by summing column E in Table I. Applying these values to Equation 2 yields non-target drug effects of 14−7 or 7. Further, in this example, the identities of the non-target drug effects are known since the microarray specifies the identity of each transcript being analyzed, indicating that transcripts X_{4,5,8,9,10,11,& 12} represent non-target drug effects relative to the model antisense response.

In one embodiment, this invention provides a a method to determine the specificity of a drug for a molecular target comprising, contacting a first cell system expressing the molecular target with a molecular target-specific compound to modulate the function of the molecular target, measuring a cellular response of the first cell system to generate a model response, contacting a second cell system substantially similar to the first cell system with a drug intended to modulate the function of the molecular target, measuring a cellular response of the second cell system to generate a drug response, where a difference between the model response and the drug is indicative of the specificity of the drug. Further, the invention provides a method for determining a drug having a higher specificity for the molecular target comprising performing the foregoing method on more than one drug, and comparing the specificity of at least two drugs to determine the drug having the higher specificity for the molecular target.

In another embodiment, this invention provides a method to identify at least one non-target effect of a drug. As used herein, a “non-target effect” or “non-intended target effect” refers to the unwanted or undesired modification of a function of a molecular target that is not the intended molecular target, or the unwanted or undesired modification of a target that is not a downstream direct or indirect result of modification of the function of the intended molecular target. The method to identify at least one non-target effect of at least one drug comprises contacting a first cell system expressing the molecular target with a molecular target-specific compound to modulate the function of the molecular target, measuring a cellular response of the first cell system to generate a model response, contacting a second cell system substantially similar to the first cell system with a drug intended to modulate the function of the molecular target, measuring a cellular response of the second cell system to generate a drug response, comparing the model response to the drug response to detect a difference between the model response and the drug response, where a difference between the model response and the drug response represents a non-target effect of the drug. As used herein, substantially similar refers to a similarity that allows for the effective use of the cell system in a specificity comparison.

In another embodiment, this invention provides a method to identify a non-target effect of a drug for a molecular target comprising contacting a first cell system not expressing the molecular target with a molecular target-specific compound to modulate the function of the molecular target, measuring a cellular response of the first cell system to generate a model response, contacting a second cell system substantially similar to the first cell system with a drug intended to modulate the function of the molecular target, measuring a cellular response of the second cell system to generate a drug response, where a difference between the model response and the drug response is indicative of a non-target effect of the drug.

In a further embodiment, this invention provides a method to identify a non-target effect of a drug for a molecular target comprising contacting a first cell system expressing the molecular target with a molecular target-specific compound and a drug to modulate the function of the molecular target, measuring a cellular response of the first cell system to generate a combined response, contacting a second cell system substantially similar to the first cell system with a target-specific agent intended to modulate the function of the molecular target, measuring a cellular response of the second cell system to generate a model response, comparing the combined response to the model response to detect a difference between the model response and the drug response, where a difference between the model response and the drug response is indicative of a non-target effect of the drug.

In a further embodiment, this invention provides a method to identify a molecular target whose function may be modulated to produce a desired biological effect. As used herein, “desired biological effect” refers to a desired or wanted cellular response of a cell system that will vary according to the molecular target and drug under investigation. As explained above, a drug often has more than one molecular target. A drug that has more than one molecular target can produce a desired biological effect. For example, a drug may inhibit the activity of a first protein. The inhibition of the first protein, in turn, may inhibit the expression of a secondary molecular target such as a second protein. The second protein may be a molecular target that itself may be modified to produce the desired biological effect without modifying the function of the first protein. The method for identifying molecular targets whose function may be modified to produce a desired biological effect comprises contacting a first cell system expressing a molecular target with a molecular target-specific compound capable of producing the desired biological effect, measuring a cellular response of the first cell system to generate a model response, contacting a second cell system expressing a second molecular target with a molecular target-specific compound to modulate the function of the second molecular target, measuring a cellular response of the second cell system, comparing the model response to the cellular response to detect molecular targets whose function has been modulated, in order to identify a molecular target whose function may be modulated to produce a desired biological effect.

In still another embodiment, this invention provides a method to refine the determination of drug specificity for a protein molecular target using a protein that is a homolog of the protein molecular target. As used herein, homolog refers to a protein in which amino acids have been deleted (e.g., a truncated version of the protein, such as a peptide), inserted, inverted, substituted and/or derivatized (e.g., by glycosylation, phosphorylation, acetylation, myristoylation, prenylation, palmitoylation, amidation and/or addition of glycerophosphatidyl inositol) such that the homolog comprises a protein having an amino acid sequence that is sufficiently similar to the molecular target that a nucleic acid sequence encoding the homolog is capable of hybridizing under stringent conditions to (i.e., with) the complement of a nucleic acid sequence encoding the corresponding molecular target amino acid sequence. As used herein, stringent hybridization conditions refer to standard hybridization conditions under which nucleic acid molecules, including oligonucleotides, are used to identify similar nucleic acid molecules. Such standard conditions are disclosed, for example, in Sambrook et al., Molecular Cloning: A Laboratory Manual, Cold Spring Harbor Labs Press, 1989; Sambrook et al., ibid., is incorporated by reference herein in its entirety. Stringent hybridization conditions typically permit isolation of nucleic acid molecules having at least about 70% nucleic acid sequence identity with the nucleic acid molecule being used to probe in the hybridization reaction. Formulae to calculate the appropriate hybridization and wash conditions to achieve hybridization permitting 30% or less mismatch of nucleotides are disclosed, for example, in Meinkoth et al., 1984, Anal. Biochem. 138, 267-284; Meinkoth et al., ibid., is incorporated by reference herein in its entirety.

A homolog may be a paralogue, in which a gene or gene product that is the result of duplication of a gene or gene product (see, e.g., Chervitz, et al., Science 1998 282:2022-28; Chervitz, et al., ibid., is incorporated by reference herein in its entirety). In one embodiment, the homolog is an orthologue. As used herein, orthologue refers to a gene or gene product in another species considered to share a common ancestor to the molecular target, see, e.g., Chervitz, et al., ibid. In another preferred embodiment, the homolog is an intraspecies homolog.

A molecular target homolog of the present invention can also be the result of allelic variation of a natural gene encoding the molecular target. A natural gene refers to the form of the gene found most often in nature. Molecular target homologs can be produced using techniques known in the art including, but not limited to, direct modifications to a gene encoding a protein using, for example, classic or recombinant DNA techniques to effect random or targeted mutagenesis. Isolated molecular targets of the present invention, including homologues, can be identified in a straight-forward manner by the molecular target's ability to effect its normal activity and/or to elicit an immune response against a molecular target. Such techniques are known to those skilled in the art. For example, a homolog of a protease molecular target will effect proteolytic activity. Additionally, when the homolog is administered to an animal as an immunogen, using techniques known to those skilled in the art, the animal will produce an immune response against at least one epitope of a natural molecular target. As used herein, the term “epitope” refers to the smallest portion of a protein or other antigen capable of selectively binding to the antigen binding site of an antibody or a T-cell receptor. It is well accepted by those skilled in the art that the minimal size of a protein epitope is about four amino acids.

The method to refine the determination of drug specificity comprises contacting a first cell system expressing the molecular target homolog with a molecular target-specific compound to modulate the function of the molecular target, wherein the function of the homolog is modulated by less than about 50%. measuring a cellular response of the first cell system to generate a model response, contacting a second cell system substantially similar to the first cell system with a drug suspected of modulating the function of the molecular target. measuring a cellular response of the second cell system to generate a drug response, comparing the model response with the drug response, in order to refine the determination of drug specificity.

In still another embodiment, this invention provides a method to determine differences in drug responses of different cell systems comprising contacting a first cell system expressing the molecular target with a molecular target-specific compound to modulate the function of the molecular target, measuring a cellular response of the first cell system to generate a model response, contacting a second cell system with a drug suspected of modulating the function of the molecular target, measuring a cellular response of the second cell system to generate a drug response, comparing the model response with the drug response to determine a difference in a cell system-specific response for the intended molecular target, where a difference in a cell system-specific response for the intended molecular target is indicative of a difference in a drug response. In a preferred embodiment, the first and second cell systems are derived from different species.

In another embodiment, this invention provides a method to determine the specificity of combinations of drugs for a molecular target or molecular targets, using combinations of drugs in the foregoing methods. or to identify at least one non-target effect as a result of the drug combination.

What is claimed is:

1. A method to determine the specificity of a drug for a molecular target comprising:;
a) contacting a first cell system expressing the molecular target with a molecular target-specific compound to modulate the function of the molecular target;
b) measuring a cellular response of the first cell system to generate a model response;
c) contacting a second cell system substantially similar to the first cell system with a drug intended to modulate the function of the molecular target;
d) measuring a cellular response of the second cell system to generate a drug response;
e) comparing the model response with the drug response, whereby the specificity of the drug for the molecular target is determined.

2. The method of claim 1, wherein the drug is a combination of more than one drug.

3. The method of claim 1 wherein the target-specific compound is an antisense reagent.

4. The method of claim 3, wherein the target-specific compound further comprises a target-specific compound selected from the group consisting of an aptamer, an antibody, a drug, a ribozyme, a zinc finger binding protein, an RNA editing protein, an siRNA, and a chimeraplast.

5. The method of claim 1, wherein the target-specific compound is selected from the group consisting of an aptamer, an antibody, a drug, a ribozyme, a zinc finger binding protein, an RNA editing protein, an siRNA, and a chimeraplast.

6. The method of claim 1, wherein measuring the cellular response comprises detecting a change selected from the group consisting of a change in cell phenotype, a change in the transcriptome, a change in metabolome, and a change in the proteome.

7. The method of claim 1, where in specificity is determined by the equation Per Cent Specificity=100%×(M)/(C).

8. A method to identify a drug with a higher specificity for a molecular target, comprising:
a) performing the method of claim 1 independently for more than one drug, and
b) comparing the specificity of at least two drugs, whereby the drug having the higher specificity for the molecular target is identified.

9. A method to identify a non-target effect of a drug for a molecular target comprising:
a) contacting a first cell system expressing the molecular target with a molecular target-specific compound to modulate the function of the molecular target;
b) measuring a cellular response of the first cell system to generate a model response;
c) contacting a second cell system substantially similar to the first cell system with a drug intended to modulate the function of the molecular target;
d) measuring a cellular response of the second cell system to generate a drug response;
e) comparing the model response to the drug response to detect a difference between the model response and the drug response, whereby a non-target effect of the drug may be identified.

10. The method of claim 9, wherein the drug is a combination of more than one drug.

11. The method of claim 9 wherein the target-specific compound is an antisense reagent.

12. The method of claim 11, wherein the target-specific compound further comprises a target-specific compound selected from the group consisting of an aptamer, an antibody, a drug, a ribozyme, a zinc finger binding protein, an RNA editing protein, an siRNA, and a chimeraplast.

13. The method of claim 9, wherein the target-specific compound is selected from the group consisting of an aptamer, an antibody, a drug, a ribozyme, a zinc finger binding protein, an RNA editing protein, an siRNA, and a chimeraplast.

14. The method of claim 9, wherein measuring the cellular response comprises detecting a change selected from the group consisting of a change in cell phenotype, a change in the transcriptome, a change in metabolome, and a change in the proteome.

15. The method of claim 9, wherein non-target drug effects are determined by the equation Non-target drug effects=C−M.

16. A method to identify a non-target effect of a drug for a molecular target comprising:
a) contacting a first cell system not expressing the molecular target with a molecular target-specific compound to modulate the function of the molecular target;
b) measuring a cellular response of the first cell system to generate a model response;
c) contacting a second cell system substantially similar to the first cell system with a drug intended to modulate the function of the molecular target;
d) measuring a cellular response of the second cell system to generate a drug response;
e) comparing the model response to the drug response to detect a difference between the model response and the drug response, whereby a non-target effect of the drug may be identified.

17. The method of claim 16, wherein the drug is a combination of more than one drug.

18. The method of claim 16, wherein the target-specific compound is an antisense reagent.

19. The method of claim 18, wherein the target-specific compound further comprises a target-specific compound selected from the group consisting of an aptamer, an antibody, a drug, a ribozyme, a zinc finger binding protein, an RNA editing protein, an siRNA, and a chimeraplast.

20. The method of claim 16, wherein the target-specific compound is selected from the group consisting of an aptamer, an antibody, a drug, a ribozyme, a zinc finger binding protein, an RNA editing protein, an siRNA, and a chimeraplast.

21. The method of claim 16, wherein measuring the cellular response comprises detecting a change selected from the group consisting of a change in cell phenotype, a change in the transcriptome, a change in metabolome, and a change in the proteome.

22. The method of claim 16, wherein non-target drug effects are determined by the equation Non-target drug effects=C−M.

23. A method to identify a non-target effect of a drug for a molecular target comprising:
a) contacting a first cell system expressing the molecular target with a molecular target-specific compound and a drug to modulate the function of the molecular target;
b) measuring a cellular response of the first cell system to generate a combined response;
c) contacting a second cell system substantially similar to the first cell system with a target-specific agent intended to modulate the function of the molecular target;
d) measuring a cellular response of the second cell system to generate a model response;
e) comparing the combined response to the model response to detect a difference between the model response and the drug response, whereby a non-target effect of the drug may be identified.

24. The method of claim 23, wherein the drug is a combination of more than one drug.

25. The method of claim 23, wherein the target-specific compound is an antisense reagent.

26. The method of claim 25, wherein the target-specific compound further comprises a target-specific compound selected from the group consisting of an aptamer, an antibody, a drug, a ribozyme, a zinc finger binding protein, an RNA editing protein, an siRNA, and a chimeraplast.

27. The method of claim 23, wherein the target-specific compound is selected from the group consisting of an aptamer, an antibody, a drug, a ribozyme, a zinc finger binding protein, an RNA editing protein, an siRNA, and a chimeraplast.

28. The method of claim 23, wherein measuring the cellular response comprises detecting a change selected from the group consisting of a change in cell phenotype, a change in the transcriptome, a change in metabolome, and a change in the proteome.

29. The method of claim 23, wherein non-target drug effects are determined by the equation Non-target drug effects=C−M.

30. A method to identify a molecular target whose function may be modulated to produce a desired biological effect comprising:
a) contacting a first cell system expressing a molecular target with a molecular target-specific compound capable of producing the desired biological effect;
b) measuring a cellular response of the first cell system to generate a model response;
c) contacting a second cell system expressing a second molecular target with a molecular target-specific compound to modulate the function of the second molecular target;
d) measuring a cellular response of the second cell system;
e) comparing the model response to the cellular response to detect molecular targets whose function has been modulated, whereby molecular targets whose function may be modulated to produce a desired biological effect may be identified.

31. The method of claim 30, wherein the drug is a combination of more than one drug.

32. The method of claim 30, wherein the target-specific compound is an antisense reagent.

33. The method of claim 32, wherein the target-specific compound further comprises a target-specific compound selected from the group consisting of an aptamer, an antibody, a drug, a ribozyme, a zinc finger binding protein, an RNA editing protein, an siRNA, and a chimeraplast.

34. The method of claim 30, wherein the target-specific compound is selected from the group consisting of an aptamer, an antibody, a drug, a ribozyme, a zinc finger binding protein, an RNA editing protein, an siRNA, and a chimeraplast.

35. The method of claim 30, wherein measuring the cellular response comprises detecting a change selected from the group consisting of a change in cell phenotype, a change in the transcriptome, a change in metabolome, and a change in the proteome.

36. A method to refine the determination of drug specificity for a protein molecular target comprising:
a) contacting a first cell system expressing the molecular target homolog with a molecular target-specific compound to modulate the function of the molecular target, wherein the function of the homolog is modulated by less than about 50%;
b) measuring a cellular response of the first cell system to generate a model response;
c) contacting a second cell system substantially similar to the first cell system with a drug suspected of modulating the function of the molecular target;
d) measuring a cellular response of the second cell system to generate a drug response;
e) comparing the model response with the drug response, whereby the determination of drug specificity may be refined.

37. The method of claim 36, wherein the drug is a combination of more than one drug.

38. The method of claim 36, wherein the target-specific compound is an antisense reagent.

39. The method of claim 38, wherein the target-specific compound further comprises a target-specific compound selected from the group consisting of an aptamer, an antibody, a drug, a ribozyme, a zinc finger binding protein, an RNA editing protein, an siRNA, and a chimeraplast.

40. The method of claim 36, wherein the target-specific compound is selected from the group consisting of an aptamer, an antibody, a drug, a ribozyme, a zinc finger binding protein, an RNA editing protein, an siRNA, and a chimeraplast.

41. The method of claim 36, wherein measuring the cellular response comprises detecting a change selected from the group consisting of a change in cell phenotype, a change in the transcriptome, a change in metabolome, and a change in the proteome.

42. A method to determine differences in drug response of different cell systems comprising:
a) contacting a first cell system expressing the molecular target with a molecular target-specific compound to modulate the function of the molecular target;
b) measuring a cellular response of the first cell system to generate a model response;
c) contacting a second cell system with a drug suspected of modulating the function of the molecular target;
d) measuring a cellular response of the second cell system to generate a drug response;
e) comparing the model response with the drug response to determine a difference in a cell system-specific response for the intended molecular target, whereby a difference in a drug response is determined.

43. The method of claim 42, wherein the drug is a combination of more than one drug.

44. The method of claim 42, wherein the first and second cell systems are derived from different species.

45. The method of claim 42, wherein the first cell system expressing the molecular target expresses a species-specific homolog of the molecular target.

46. The method of claim 42, wherein the target-specific compound is an antisense reagent.

47. The method of claim 46, wherein the target-specific compound further comprises a target-specific compound selected from the group consisting of an aptamer, an antibody, a drug, a ribozyme, a zinc finger binding protein, an RNA editing protein, an siRNA, and a chimeraplast.

48. The method of claim 42, wherein the target-specific compound is selected from the group consisting of an aptamer, an antibody, a drug, a ribozyme, a zinc finger binding protein, an RNA editing protein, an siRNA, and a chimeraplast.

49. The method of claim 42, wherein measuring the cellular response comprises detecting a change selected from the group consisting of a change in cell phenotype, a change in the transcriptome, a change in metabolome, and a change in the proteome.
